(19) United States
(12) Patent Application Publication
(10) Pub. No.: US 2015/0265161 A1
(43) Pub. Date: Sep. 24, 2015

(54) Methods and Apparatus for Physiological Parameter Estimation

(71) Applicants: Javier Hernandez, Cambridge, MA (US); Yin Li, Atlanta, GA (US); James Rehg, Atlanta, GA (US); Rosalind Picard, Newton, MA (US)

(72) Inventors: Javier Hernandez, Cambridge, MA (US); Yin Li, Atlanta, GA (US); James Rehg, Atlanta, GA (US); Rosalind Picard, Newton, MA (US)

(73) Assignee: MASSACHUSETTS INSTITUTE OF TECHNOLOGY, Cambridge, MA (US)

(21) Appl. No.: 14/661,747

(22) Filed: Mar. 18, 2015

Related U.S. Application Data
(60) Provisional application No. 61/955,772, filed on Mar. 19, 2014.

(51) Int. Cl. A61B 5/0205; A61B 5/024; A61B 5/00; A61B 5/08; A61B 5/11

(52) U.S. Cl. CPC A61B 5/0205; A61B 5/08; A61B 5/1123; A61B 5/0077; A61B 5/024

(57) ABSTRACT

In illustrative implementations, a gyroscope, an accelerometer and a camera gather sensor data indicative of motion of a human head. The gyroscope, accelerometer and camera are each housed in, or attached to, headwear that is worn on the head. In some cases, the headwear comprises a headband, hat, cap, or structure similar to an eyeglasses frame. A computer takes the sensor data as input and calculates a heart rate and respiration rate of the human. In some cases, a computer also calculates heart rate variability. The head motion being measured is caused by forces that are transmitted, at least in part, from the chest, through the neck, and to the head. This head motion is caused, at least in part, by respiration, by heart beats, or by blood flow caused by the heart beats.

### RELATED APPLICATIONS

This application is a non-provisional of, and claims the benefit of the filing date of, U.S. Provisional Patent Application No. 61/955,772, filed Mar. 19, 2014, the entire disclosure of which is herein incorporated by reference.

### STATEMENT REGARDING FEDERALLY SPONSORED RESEARCH OR DEVELOPMENT

This invention was made with government support under Grant No. IIS-1029585 awarded by the National Science Foundation. The government has certain rights in the invention.

### FIELD OF TECHNOLOGY

The present invention relates generally to head-mounted sensors for measurements of respiration rate, heart rate, or heart rate variability.

### SUMMARY

In illustrative implementations of this invention, a gyroscope, an accelerometer and a camera gather sensor data indicative of motion of a human head. The gyroscope, accelerometer and camera are each housed in, or attached to, headwear that is worn on the head. In some cases, the headwear comprises a headband, hat, cap, or structure similar to an eyeglasses frame. A computer takes the sensor data as input and calculates a cardiac pulse rate and respiration rate of the human. In some cases, a computer also calculates heart rate variability. The head motion being measured is caused by forces that are transmitted, at least in part, from the chest, through the neck, and to the head. This head motion is caused, at least in part, by respiration, by heart beats, or by blood flow caused by the heart beats.

In illustrative implementations, a head-mounted sensor module includes at least three sensors: a tri-axial gyroscope, a tri-axial accelerometer, and a camera. The sensor module measures respiration rate and heart rate of a human user who is wearing the head-mounted sensor.

The sensor module is positioned such that the camera faces forward and captures a field of view that overlaps with the field of view of the human user. For example, in some cases, the sensor module is worn above the right eye of a human user, or on any other position on the forehead. In some cases, the sensor module is housed in a support structure that is similar in shape to, or is part of, of an eyeglasses frame. In some cases, the sensor module is attached to a head band, sweat band or other stretchy band that is worn on a human's head.

A prototype of this invention has been evaluated in a test with human subjects.

Test data gathered during this evaluation shows that a combination of measurements from all three sensors yields more accurate measurements of respiration rate than measurements from any of the three sensors alone.

Also, the test data shows that the gyroscope alone takes the most accurate measurement of heart rate, compared to measurements of heart rate taken by the accelerometer alone, by the camera alone, or by a combination of the three sensors.

An advantage of positioning a gyroscope in a head-mounted sensor is that rotational movements caused by heart beat and respiration are amplified by the head's placement atop a flexible neck. In contrast, these rotational movements tend to be smaller on the main torso of the user (where they are not amplified by the neck).

In some cases, the gyroscope is housed in a sensor module that is positioned on the forehead of a user. An advantage of positioning a gyroscope on the forehead is that rotational movements caused by heart beat and respiration are more amplified at the forehead than at a position behind the ear (such as at skin covering the mastoid process).

Conventional ballistocardiographic techniques measure linear motions and linear acceleration, and do not measure rotational movements. For example, an early ballistocardiographic study involved (a) a patient lying on a bed that is free to move, with very little friction, in linear directions parallel to the floor, and (b) measuring linear movements of the bed, caused by beats of the patient's heart.

In contrast, in illustrative implementations, a gyroscope measures rotational movements of the head, caused by heart beats, blood flow from heart beats, or respiration.

Advantageously, the data gathered by the three sensors in the sensor modules provide contextual information. For example, video images captured by the camera in the head-mounted sensor module may provide information regarding whether increased heart rate is due to a stressful event (such as giving a speech) or due instead to exercise. Likewise, accelerometer data may indicate that the user is exercising.

In illustrative implementations, the sensor module includes sensors that measure different things (i.e., a gyroscope measures rotation, an accelerometer measures linear acceleration, and the camera captures visual images). Having a sensor module with different sensors that measure different things is advantageous, because in some use scenarios, large artifacts reduce the accuracy of one or two of the sensors, but not the remaining sensor(s). For example, in some cases, a computer disregards, for purposes of calculating respiration rate, cardiac pulse rate or heart rate variability, data gathered by the accelerometer during periods in which the magnitude of acceleration measured by the accelerometer exceeds a specified threshold. For example, in a rapidly accelerating car, the car's acceleration produces a large artifact for the accelerometer, but does not affect the gyroscope. In that case, it may be desirable to disregard the accelerometer data gathered during the rapid acceleration of the car.

The description of the present invention in the Summary and Abstract sections hereof is just a summary. It is intended only to give a general introduction to some illustrative implementations of this invention. It does not describe all of the details of this invention. This invention may be implemented in many other ways. Likewise, the description of this invention in the Field of Technology section is not limiting; instead it identifies, in a general, non-exclusive manner, a field of technology to which exemplary implementations of this invention generally relate.

### BRIEF DESCRIPTION OF THE DRAWINGS

FIG. 1 is a conceptual diagram that shows an overview of hardware and methods for determining respiration rate, heart rate and heart rate variability.

FIG. 2 is a flowchart showing steps in a method for determining heart rate and heart rate variability.

FIG. 3 is a flowchart showing steps in a method for determining respiration rate.

FIG. 4 is a flowchart showing steps in extracting physiological data from a motion video.

FIG. 5 is a perspective view of an example of a head-mounted sensor module and support structure.

FIG. 6A is a top view of another example of a head-mounted sensor and support structure.

FIG. 6B shows computer-readable media.

FIG. 7A is a top view of overlapping fields of view.

FIG. 7B is a side view that illustrates a camera adjacent to a face.

The above Figures show some illustrative implementations of this invention. However, this invention may be implemented in many other ways.

### DETAILED DESCRIPTION

In exemplary implementations of this invention, a head-mounted sensor module includes at least three sensors: a tri-axial gyroscope, a tri-axial accelerometer and a camera. The sensor module measures respiration rate, heart rate and heart rate variability of a human user who is wearing the head-mounted sensor.

The sensor module is positioned such that the camera faces forward and captures a field of view that overlaps with the field of view of the human user.

### Prototype

The following seven paragraphs are a description of a prototype of this invention. The prototype is a non-limiting example of this invention. This invention may be implemented in many other ways.

In this prototype, a sensor module is worn above the right eye of a human user. The sensor module is housed in a support structure that resembles, in size and shape, the frame for a pair of eyeglasses. The sensor module includes a 3-axis gyroscope, 3-axis accelerometer and a video camera. A computer that is housed in the support structure executes a program that simultaneously logs information from the accelerometer, the gyroscope and the camera of the sensor module.

In this prototype, the 3-axis accelerometer captures acceleration (meters/second) along X, Y and Z axes. The 3-axis gyroscope captures the rate of rotation (radians/second) of the device about X, Y and Z axes. The gyroscope and accelerometer each take samples at an average rate of 50 Hz. For both the gyroscope and accelerometer data, a computer housed in the support structure (a) performs cubic interpolation at a sampling rate of sampled data at 256 Hz, and (b) applies a hard-thresholding algorithm (disregarding data that is more 2 STD above or below the mean) to reduce sensor artifacts and remove large motion during each observation window.

In this prototype, the camera records video at a constant frame rate of 30 Hz at a resolution of 1280×720 pixels. Each of the pixels yields a vector in RGB color space. A computer estimates motion of the sensor module by tracking 2D feature points in the video. The computer performs a motion estimation algorithm that includes the following steps. First, detect feature points in each frame and track them using a Kanade-Lucas-Tomasi feature tracker method. Second, fit a homography matrix to the point correspondences using RANSAC (random sample consensus). Third, extract vertical and horizontal motion (up to a scale) of the camera from the homography matrix. This algorithm assumes that all tracked points correspond to static 3D points, in which case their offsets are solely explained by the camera motion.

In this prototype, a computer determines a heart pulse wave by performing an algorithm that includes the following steps: First, represent the sensor data as a time series of vectors (e.g., 3D vector for accelerometer and gyroscope, 2D vector for camera). Second, subtract a moving average window of 3 samples from each dimension of the vector, allowing the removal of signal shifts and trends. Third, apply a fourth-order Butterworth high-pass filter with a cut-off frequency of 10 Hz and a fourth-order Butterworth low-pass filter with a cut-off frequency of 13 Hz to each dimension. Fourth, in order to aggregate the different components of the signal (i.e. dimensions of the vector), compute the square root of the summation of the squared components (i.e., L2 norm) at each sample. This aggregation gives the same relevance to each of the dimensions and makes the output more robust to different body postures. Fifth, apply a second-order Butterworth high-pass filter with a cut-off frequency of 0.75 Hz and a second-order Butterworth low-pass filter with a cut-off frequency of 2.5 Hz to each dimension, yielding a heart pulse wave. The cut-off frequencies correspond to 45 and 150 beats per minute.

In this prototype, a computer determines a respiration wave by performing an algorithm that includes the following steps: First, represent the sensor data as a time series of vectors (e.g., 3D vector for accelerometer and gyroscope, 2D vector for camera). Second, apply an average filter to each of the components of the signal (i.e. dimensions of the vector) with a window length equal to the duration of a respiration cycle at a breathing rate of 45 breaths per minute. This filter removes cardiac changes that are in a higher frequency range. Third, apply a fourth-order Butterworth high-pass filter with a cut-off frequency of 0.13 Hz and a fourth-order Butterworth low-pass filter with a cut-off frequency of 0.75 Hz are computationally applied to each dimension. The cut-off frequencies correspond to 8 and 45 breaths per minute.

In this prototype, a computer uses PCA (principal component analysis) to determine principal components of the respiration wave. (PCA is performed because different dimensions of the sensor readings (e.g., X and Y axes of accelerometer) often change in different directions and PCA transforms the data into principal components that maximize the variance. After the PCA, a computer calculates a Fast Fourier Transform of each principal component and selects the principal component with the maximum amplitude observed within a band of frequencies (e.g., 0.13-0.75 Hz for respiration rate).

In this prototype, a computer performs an algorithm that extracts heart rate and the respiration rate in the frequency domain. The algorithm takes estimated pulse and respiratory waves as input. The algorithm involves extracting the frequency response with the Fast Fourier Transform and identifying the frequency with the highest amplitude response. In this algorithm, the band of frequencies used for the pulse and respiration rates are 0.75-2.5 Hz for heart rate and 0.13-0.75 Hz for respiration rate. In this algorithm, the final estimated heart rate and respiration rate are equal to the maximum frequency multiplied by 60 (beats or breaths per minute). Computing these parameters in the frequency domain instead of the time domain has several advantages, including: (a) mitigating a problem of missing peaks due to non-constant sampling rates of accelerometer and gyroscope, (b) handling non-linear phase responses of the Butterworth filter, and (c) avoiding the need for peak detection in the time domain.

The prototype described in the preceding seven paragraphs is a non-limiting example of an implementation of this invention. This invention may be implemented in many other ways.

### Evaluation of Prototype

An experiment was performed to evaluate the accuracy of the measurements of the sensor unit in the prototype described above. Twelve participants (6 females) with an average age of 27.3 (STD of 5.3) years old, weight of 144.5 (STD: 30.9) pounds and height of 5.65 (STD: 0.4) feet participated in this experiment. Participants were asked to keep still, breathe spontaneously and look at a static indoor scene situated at a distance of 2.2 meters while remaining in three different positions (standing up, sitting down and lying down) for a minute each. In order to generate a larger dynamic range of physiological readings, participants were then asked to repeat the three positions after pedaling a stationary bike for one minute. Each of the 12 participants held three different positions under relaxed and aroused (after biking) conditions for a minute each. Therefore, in this experiment, 72 1-minute segments of data were collected. In order to increase the number of samples, the data was divided into intervals of 20 seconds with a 75% overlap, yielding 648 samples.

The following tables summarize results of the experiment:

TABLE I
HEART RATE ESTIMATION

| Sensor | ME | STD | RMSE | CC |
|---|---|---|---|---|
| Gyroscope | 0.82 | 1.98 | 2.14 | 0.99 |
| Accelerometer | 2.51 | 7.03 | 7.46 | 0.91 |
| Camera | 7.92 | 13.4 | 15.56 | 0.58 |
| All | 1.19 | 3.42 | 3.62 | 0.98 |

TABLE II
RESPIRATION RATE ESTIMATION

| Sensor | ME | STD | RMSE | CC |
|---|---|---|---|---|
| Gyroscope | 1.39 | 2.27 | 2.66 | 0.75 |
| Accelerometer | 2.29 | 3.43 | 4.12 | 0.41 |
| Camera | 1.55 | 2.59 | 3.02 | 0.69 |
| All | 1.16 | 2.04 | 2.35 | 0.79 |

In these two tables: “ME” is mean absolute error; “STD” is standard deviation of the absolute error; “RMSE” is root mean squared error; and CC is Pearson's correlation coefficient.

The data gathered in this experiment (and summarized in the above tables) shows that: When comparing the accuracy of the three sensors individually (i.e., comparing the accuracy of the gyroscope alone, the accuracy of the accelerometer alone, and the accuracy of the camera alone), the gyroscope had the most accurate measurement for both heart and respiration rates, achieving a mean absolute error of 0.82 beats per minute (STD: 1.98) and 1.39 breaths per minute (STD: 2.27), respectively.

The data gathered in this experiment (and summarized in the above tables) also shows that: Respiration rate is estimated more accurately based on a combination of sensor readings by all three sensors (gyroscope, accelerometer and camera) than based on sensor readings from any of the three sensors alone, achieving a mean absolute error of 1.16 breaths per minute (STD 2.04).

Thus, test data gathered during this evaluation (and summarized in Table II above) shows that a combination of measurements from all three head-mounted sensors (gyroscope, accelerometer and camera) yields more accurate measurements of respiration rate than measurements from any of the three sensors alone. The combined measurement of all three sensors (gyroscope, accelerometer and camera) is labeled “All” in Tables I and II above. This combined measurement was computed as follows: A computer calculated a separate respiration rate for each modality (gyroscope, accelerometer and camera) and then computed the median of the separate respiration rates. This median was used as the combined measurement of respiration rate. Similarly, a computer calculated a separate heart rate for each modality (gyroscope, accelerometer and camera) and then computed the median of the separate heart rates. This median was used as the combined measurement of heart rate.

Also, the test data gathered during this evaluation (and summarized in Table I above) shows that the gyroscope alone takes the most accurate measurement of cardiac pulse rate, compared to measurements of cardiac pulse rate taken by the accelerometer alone, by the camera alone, or by a combination of the three sensors.

### More Details

This invention is not limited to the prototype described above. Among other things:

In some implementations of this invention, a computer also computes heart rate variability. The heart rate variability is a statistical measure of variability of beat-to-beat (also called “NN”) intervals in the heart pulse wave.

A wide range of statistical measures may be used for calculating heart rate variability. For example, in some cases, heart rate variability is measured by one or more of: (a) SDNN, the standard deviation of NN intervals; (b) SDSD, the standard deviation of successive differences between adjacent NNs; (cc) NN50, the number of pairs of successive NNs that differ by more than 50 ms; (d) pNN50, the proportion of NN50 divided by total number of NNs; (e) NN20, the number of pairs of successive NNs that differ by more than 20 ms.; and (f) pNN20, the proportion of NN20 divided by total number of NNs.

In some implementations, in order to determine heart rate variability, a computer calculates beat-to-beat intervals in the time domain, by recognizing amplitude peaks in the heart pulse wave and determining time intervals between the amplitude peaks.

In other implementations, heart rate variability is calculated in the Fourier frequency domain. For example, in some cases, a computer assigns high and low bands of frequency (typically 0.04-0.12 and 0.15-0.4 Hz) and computes the area under the curve of the corresponding power spectral density estimation. In some cases, ratios between these areas are computed to capture different aspects of cardiac functioning. For example, in some cases, the area under low band divided by the area under the high band is measured and treated as a proxy for sympatho/vagal balance or to reflect sympathetic modulations. In some cases, a computer calculates power distribution over different frequencies, by using DFT (discrete Fourier transform), PSD (power spectral density), a Lomb-Scargle periodogram, or a wavelet entropy measure.

In some cases, the sensor module includes a gyroscope, accelerometer and camera. In other cases, the sensor module includes one or two of these three sensors, but not the remainder of these three sensors. For example: (a) in some cases, the sensor module includes a gyroscope but not an accelerometer or camera; (b) in some cases, the sensor module includes a camera but not a gyroscope or accelerometer; (c) in some cases, the sensor module includes an accelerometer but not a gyroscope or camera; (d) in some cases, the sensor module includes a gyroscope and an accelerometer but not a camera; (e) in some cases, the sensor module includes a gyroscope and camera but not an accelerometer; and (f) in some cases, the sensor module includes an accelerometer and camera but not a gyroscope.

This invention is not limited to just these three types of sensors. In some cases, the sensor module includes other types of sensors, in addition to one or more of a gyroscope, accelerometer and camera.

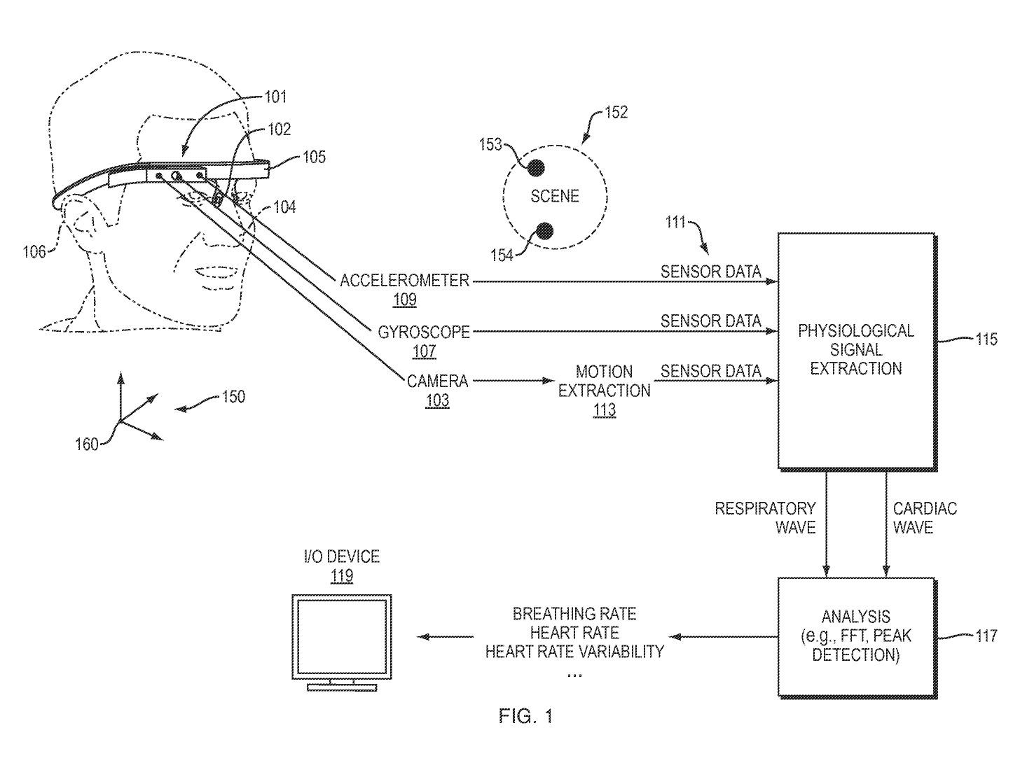

FIG. 1 is a conceptual diagram that shows an overview of hardware and methods for determining respiration rate, heart rate and heart rate variability, in an illustrative implementation of this invention. A sensor module 101 is positioned adjacent to the face or head of a user, such that a camera 103 onboard the sensor module 101 captures a field of view that overlaps with the user's field of view. The sensor module 101 is housed in, or permanently or releasably attached to, a support structure 105. The support structure 105 is configured to be worn on or over the face or head of the user. The support structure includes two nosepads (including nosepad 102) that rest on the nose 104 of the user, and includes regions (similar to the “temples” or “earpieces” of eyeglasses frames) that rest on the ears (e.g., 106) of the user.

In the example shown in FIG. 1, the sensor module 101 includes a tri-axial gyroscope 107, tri-axial accelerometer 109, and a video camera 103. The gyroscope, accelerometer and video camera gather sensor data (Step 111). A computer analyzes the video feed to determine motion of the camera relative to the scene 152 imaged by the camera. Most motion is assumed to be due to head movements, instead of movements of objects (e.g., 153, 154) in the scene. (Step 113) A computer analyzes rotational motion measurements from the gyroscope 107, linear acceleration measurements taken by the accelerometer 109, and motion data extracted from the camera's 103 video feed, in order to extract physiological signals. These signals include a cardiac pulse wave and a respiration wave (Step 115). A computer analyzes these signals to determine breathing rate, heart rate and heart rate variability (Step 117). One or more I/O devices 119 output, in human readable form, at least the heart rate and the respiration rate calculated by the computer. In some cases, the one or more I/O devices 119 also output, in human readable form, the heart rate variability calculated by the computer.

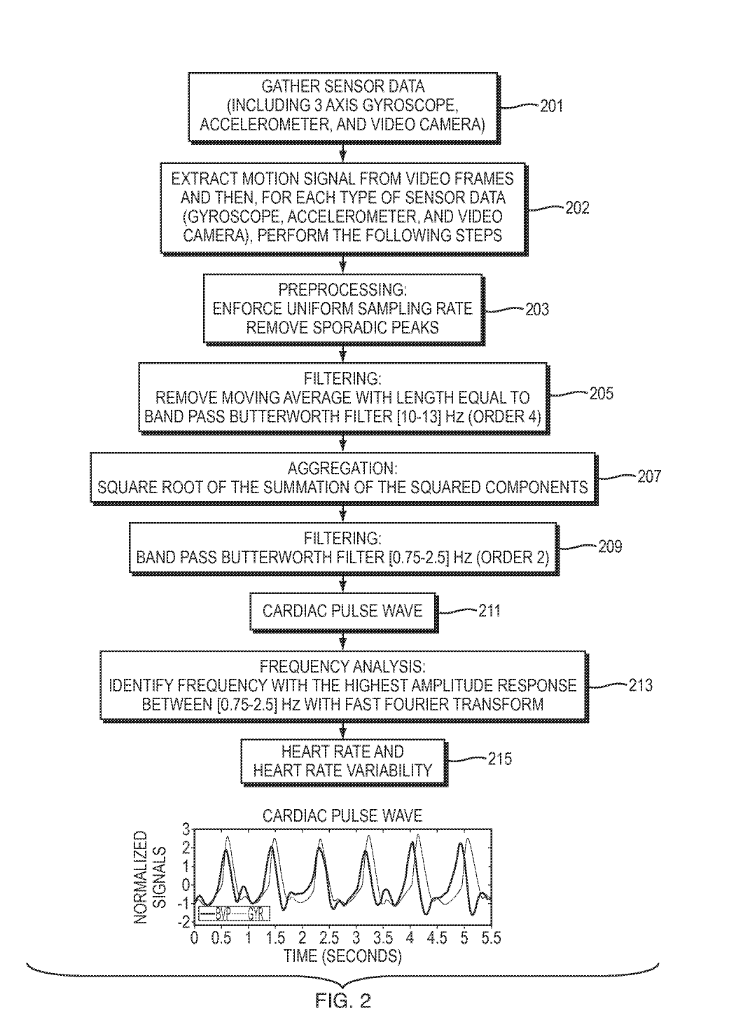

FIG. 2 is a flowchart showing steps in a method for determining heart rate and heart rate variability, in an illustrative implementation of this invention. In the method shown in FIG. 2, a computer takes, as input, multidimensional sensor data, including measurements taken by a gyroscope, accelerometer and video camera, each of which are head-mounted (step 201). The computer extracts a motion signal from video frames captured by the camera (step 202). Then, for each type of sensor data, respectively (e.g., gyroscope, accelerometer, camera), a computer: (a) performs preprocessing to enforce a uniform sampling rate and to remove sporadic peaks (step 203); (b) filters by removing a moving average and computationally applying a fourth-order Butterworth bandpass filter with cutoff frequencies of 10 Hz and 13 Hz (step 205); (c) performs aggregation by calculating a square root of the summation of squared components (step 207); (d) filters by computationally applying a fourth-order Butterworth bandpass filter with cutoff frequencies of 0.75 Hz and 2.5 Hz (step 209); (e) calculates a cardiac pulse wave (step 211); (f) calculates a Fast Fourier Transform and identifies the frequency with the highest amplitude response in the 0.75 Hz to 2.5 Hz frequency range (step 213); and (g) calculates heart rate and heart rate variability (step 215).

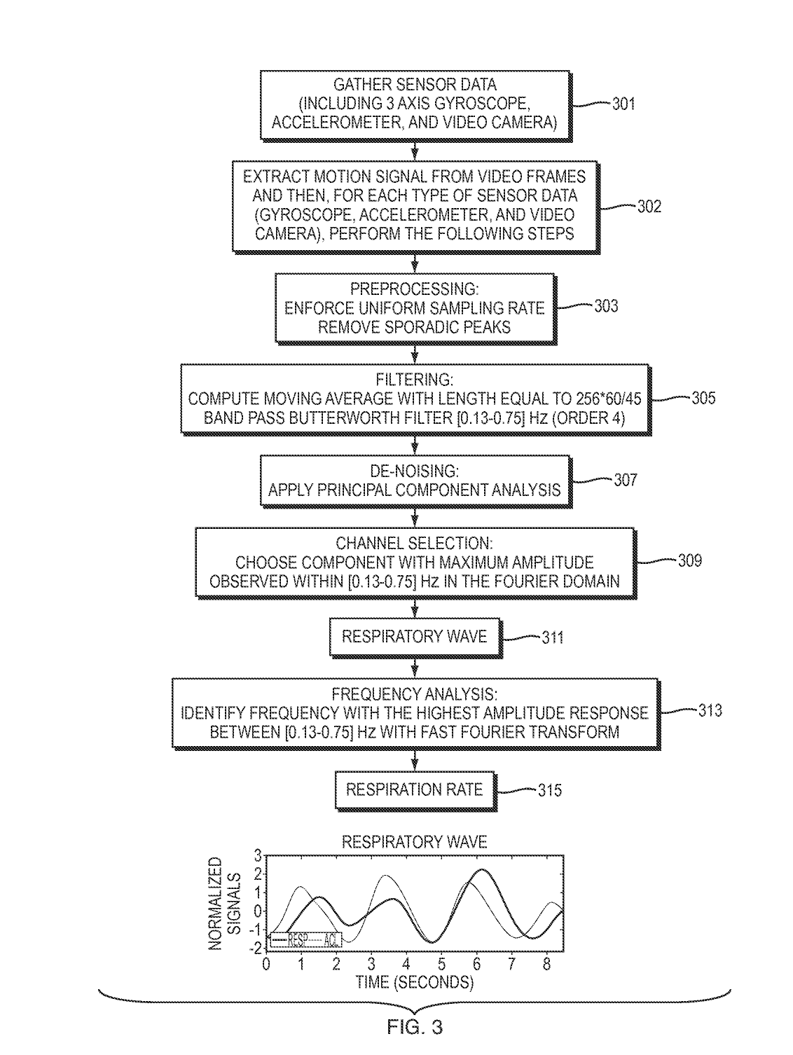

FIG. 3 is a flowchart showing steps in a method for determining respiration rate, in an illustrative implementation of this invention. In the method shown in FIG. 3, a computer takes, as input, multidimensional sensor data, including measurements taken by a gyroscope, accelerometer and video camera, each of which are head-mounted (step 301). The computer extracts a motion signal from video frames captured by the camera (step 302). Then, for each type of sensor data, respectively (e.g., gyroscope, accelerometer, camera), a computer: (a) performs preprocessing to enforce a uniform sampling rate and to remove sporadic peaks (step 303); (b) performs filtering to remove a moving average and to computationally apply a fourth-order Butterworth bandpass filter with cutoff frequencies of 0.13 Hz and 0.75 Hz (step 305); (c) denoises by performing principal component analysis (step 307); (d) selects a channel by choosing a component with the maximum amplitude observed within the 0.13 Hz to 0.75 Hz range in the Fourier frequency domain (step 309); (e) calculates a respiration wave (step 311); (g) calculates a Fast Fourier Transform and identifies the frequency with the highest amplitude response in the 0.13 Hz to 0.75 Hz frequency range (step 313); and (f) calculates respiration rate (step 315).

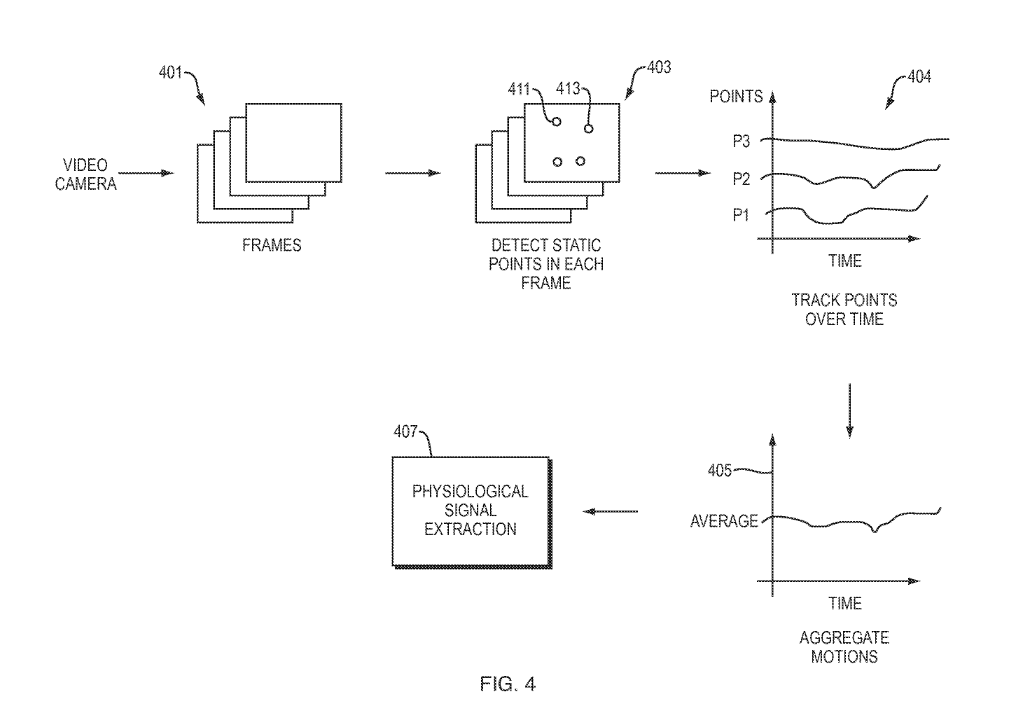

FIG. 4 is a flowchart showing steps in extracting physiological data from a motion video, in an illustrative implementation of this invention. The method in FIG. 4 includes the following steps: First use a video camera to capture multiple frames of a video (Step 401). Then, use a computer: (a) to analyze the frames to detect points (e.g., 411, 413) in each frame that are not moving relative to other points in the scene, but that may be moving relative to the video camera (step 403); (b) to track the position of the points over time (to detect apparent displacement of the points due to motion of the user's head) (step 404); (c) to calculate an average of the apparent displacement of the points (step 405); and (c) to analyze the average apparent displacement to calculate a physiological signal, such as a respiration wave or cardiac pulse wave (step 407). In the method shown in FIG. 4, apparent motion of points in a scene is assumed to be actual movements of the head-mounted camera (and thus of the head). The actual movements are relative to a spatial coordinate system (e.g., 150). The origin 160 of the spatial coordinate system is not fixed with respect to the camera or points in the scene being imaged by the camera. Thus, the distance between the origin 160 and the camera or the points in the scene may vary if the camera or points in the scene move relative to the origin 160.

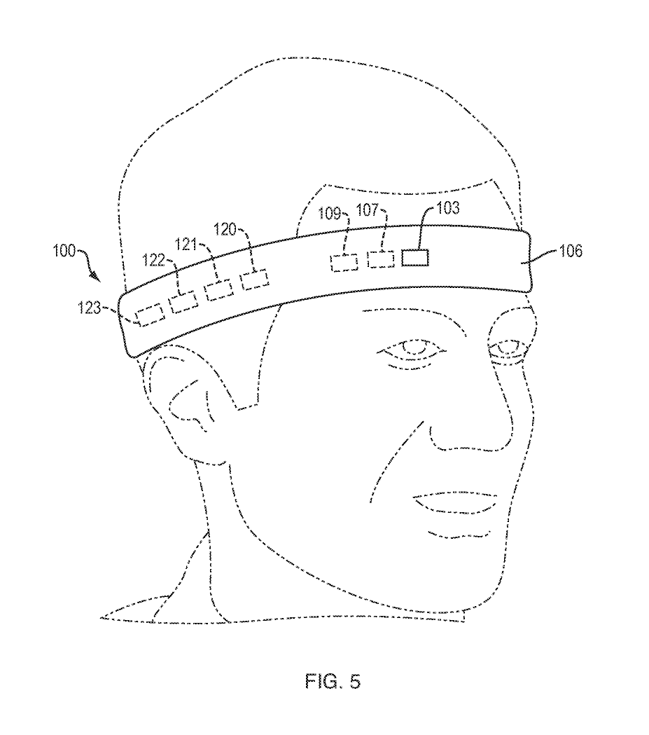

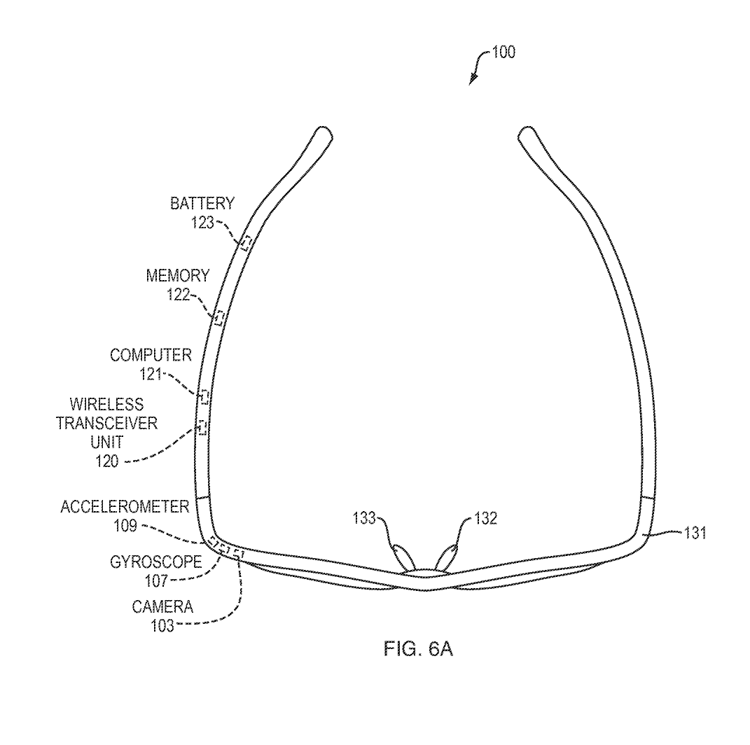

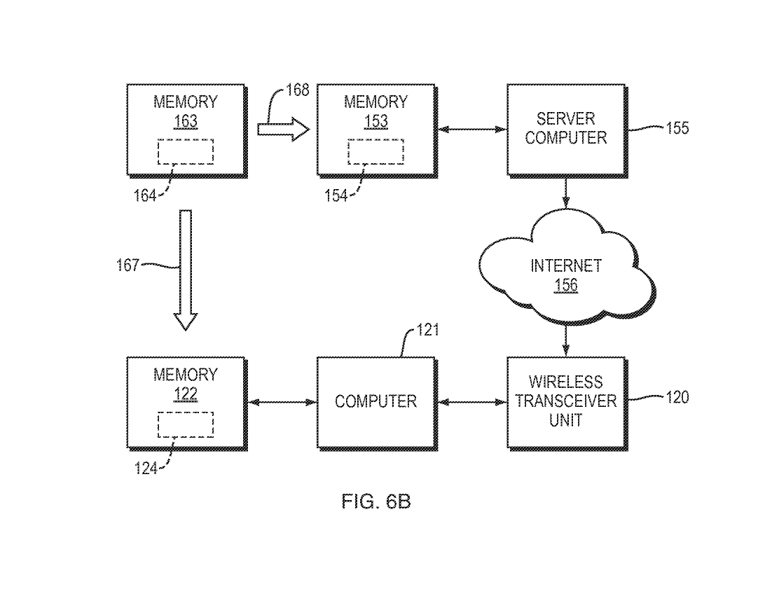

FIGS. 5 and 6A show illustrative implementations of this invention. FIG. 5 is a perspective view of an example of a head-mounted sensor module and support structure. FIG. 6A is a top view of another example of a head-mounted sensor module and support structure. In FIGS. 5 and 6A, the sensor module includes a gyroscope, accelerometer and camera.

In the examples shown in FIGS. 5 and 6A, a gyroscope 107, accelerometer 109, camera 103, wireless transceiver unit 120, computer 121, memory device 122 and battery 123 are housed in, or permanently or releasably attached to, a support structure.

A wide variety of cameras may be used. For example, in some cases, the camera is a video camera. In some cases, the camera is a depth-sensing camera, including a depth-sensing video camera.

A wide variety of support structures may be used.

In the example shown in FIG. 5, the support structure comprises elastic headware 106. In some cases, the elastic headwear 106 comprises a material that stretches (elastically deforms). In some cases, this headwear 106, when elastically deformed, has a length, around a circumference or perimeter of the headware (or around the edge of a hole formed by the headware) that: (a) is in a range between 50 cm and 65 cm, and thus is configured to fit snugly around an adult human head; or (b) is in a range between 40 cm and 55 cm, and thus is configured to fit snugly around a child's head; or (c) is in a range between 32 cm and 52 cm, and thus is configured to fit snugly around a head of a human who is between zero and 36 months old. For example, in some cases, the elastic headwear 106 comprises (i) a headband, or (ii) elastic apparel that has a convex shape that fits on or over (or partially surrounds or conforms to the shape of) a human head.

More generally, the support structure comprises any headwear, including: (a) any hat, cap, helmet, eyeglasses frame, sunglasses frame, visor, headband, crown, diadem, or head-mounted display, or (b) any structure (including any strap, band, frame, ring, post, scarf, or other item of apparel) that is worn at least partially on or supported at least partially by the skin, hair, nose or ears of a human head or that at least partially surrounds or indirectly rests upon a human neurocranium. However, the term “headwear” does not include any part of a human being.

In the example shown in FIG. 6A, at least a portion of support structure 131 is rigid. In some cases, support structure 131 includes joints or hinges, such that rigid portions of structure 131 may rotate about the joint or hinge. Support structure 131 is configured to rest upon protuberances of a human head. Specifically, support structure 131 is configured to rest upon, and be supported by, the ears and nose of a human user. For example, support structure 131 includes two nosepads 132, 133. Support structure 131 is similar in shape to, or is part of, of an eyeglasses frame.

In the physiological parameter measurement system 100 shown in FIGS. 5 and 6A, a computer 121 processes sensor data gathered by the gyroscope 107, accelerometer 109 and video camera 103. A computer (e.g., computer 121 or a remote computer) uses this sensor data to calculate respiration rate, heart rate and heart rate variability. In some cases, the computer 121 comprises a microprocessor. The computer 121 stores data in, and reads data from, the memory device 122. The computer 121 communicates with remote devices via a wireless transceiver unit 120. The wireless transceiver unit 120 includes (a) one or more antennas, (b) one or more wireless transceivers, transmitters or receivers, and (c) signal processing circuitry. The wireless transceiver unit 120 receives and transmits data in accordance with one or more wireless standards. The battery 123 provides power for the sensors (including gyroscope, accelerometer, and video camera), computer, memory device, and wireless transceiver unit.

In some cases, one or more tangible, non-transitory machine-readable media are employed. Each machine-readable medium stores instructions for a program for determining heart rate, respiration rate or heart rate variability. The program takes, as input, sensor data gathered by a gyroscope, accelerometer, or camera worn on a human head (e.g., on the forehead). The program calculates heart rate, respiration rate or heart rate variability. In the example shown in FIG. 6B, the machine-readable media 124, 154, 164 store identical copies of this program. Thus, each of the machine-readable media 124, 154, 164 stores the encoded instructions for this program.

FIG. 6B illustrates three examples of machine readable-media that store the program.

In FIG. 6B, machine-readable medium 124 is part of memory device 122, which is housed in support structure 106 or 131.

In FIG. 6B, machine-readable medium 154 is part of memory device 153, which is part of, or auxiliary to, server computer 155. Server computer 155 is connected to the Internet 156. In some cases, the program is downloaded from the server computer via the Internet 156. For example, in some cases, the download involves transferring a copy of the encoded program instructions from machine-readable medium 154 to server computer 155, then over the Internet 156 to wireless transceiver unit 120, then to computer 121, and then to machine-readable medium 124, which is part of memory device 122.

In FIG. 6B, machine-readable medium 164 comprises all or part of a memory device 163. For example, in some cases, machine-readable medium 164 stores a master copy or backup copy of the encoded program instructions. In some cases, the program instructions encoded in the master copy are copied 167 into machine-readable medium 124 during manufacturing of physiological parameter measurement system 100. In some cases, the program instructions encoded in the master copy are copied 168 into machine-readable medium 154, which is used in downloading the program, as discussed above.

In some cases, a machine-readable medium (e.g., 124, 154, or 164) comprises part or all of an electronic memory storage device, such as a RAM (random-access memory), DRAM (dynamic random-access memory), ROM (read only memory), PROM (programmable read only memory), EPROM (erasable programmable read only memory), or EEPROM (electrically erasable programmable read only memory) device; and (b) the program is encoded in voltage levels in a set of electronic components (e.g., flip-flops or latches) in the medium. In some cases: (a) voltage levels in hardware components of the machine-readable medium encode a set of logic states that do not change throughout an entire time interval that has a non-zero duration, and (b) the hardware components of the machine-readable medium exist throughout this entire time period. Alternatively, a machine-readable medium (e.g., 124, 154, or 164) comprises part or all of a CD-ROM or other optical disc storage device, and a computer reads data or instructions stored in the CD-ROM by using an optical disc driver.

A wide variety of algorithms may be used to process the sensor data to calculate respiration rate, heart rate and heart rate variability. In some cases, in order to calculate respiration rate, heart rate or heart rate variability, a computer (e.g., computer 121 or a remote computer) performs one or more of the algorithms that are: (a) described in FIGS. 1, 2, 3 and 4 and accompanying text of this document; or (b) otherwise described above in this document. In some cases, in order to calculate heart rate, a computer (e.g., computer 121 or a remote computer) performs one or more of the following algorithms: (a) signal segmentation with template beat wave matching; (b) adaptive beat to beat estimation based on component analyses; (c) a neural network algorithm, (d) an algorithm that uses a statistical autocorrelation function, or signal energy thresholding, or peaks in a signal energy envelope, in order to compute heart rate.

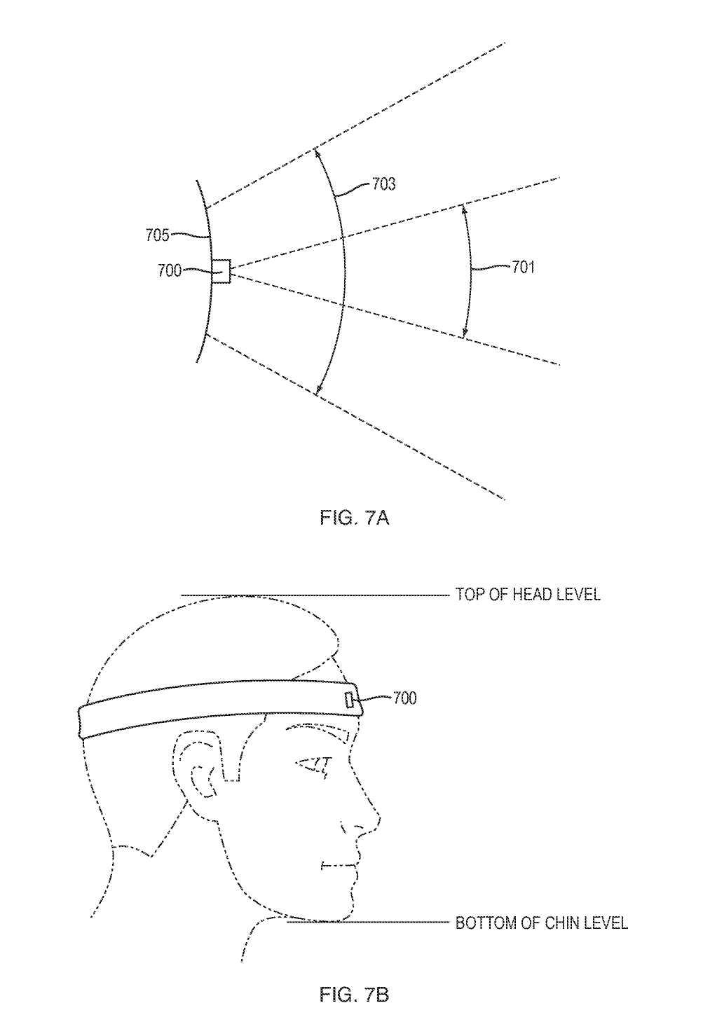

FIG. 7A is a top view of overlapping fields of view. In the example shown in FIG. 7A, a camera 700 is positioned such that: (a) the camera's field of view 701 overlaps the user's field of view 703; and (b) the camera is adjacent to the user's face 705. This positioning of the camera 700 is achieved by selecting an appropriate size and shape of the support structure and an appropriate position, on the support structure, for housing or attaching the camera to the support structure. Having an overlapping field of view is advantageous because the camera captures images of at least part of the scene viewed by the user, and thus may record data regarding a visual context that is seen by the human user. The user's reaction to the visual context may affect respiration rate, heart rate and heart rate variability.

Alternatively, the camera's field of view does not overlap the user's field of view. Alignment of the camera with the user's field of view is not necessary in order for the camera to capture head motions from which heart rate, respiration rate and heart rate variability are extracted. A camera that is touching the user's head (or attached to a structure touching the user's head that transmits motion from the head to the camera) undergoes movements due to heart beats, blood flow from heart beats and respiration of the user. These movements are detectable in video images captured by the camera.

FIG. 7B is a side view that illustrates a camera adjacent to a face. In the example shown in FIG. 7B, the camera 700 is at a vertical level that is at or above the bottom of the chin of the user and at or below the top of the head of the user. This vertical positioning tends to align the camera's field of view with the user's field of view.

In illustrative implementations, a computer performs an algorithm for calculating respiration rate. The algorithm includes applying one or more filters to an input signal derived from the sensor data and producing a filtered signal, such that the overall effect of the one of more filters is a bandpass filter. The bandpass filter has a first cutoff frequency and a second cutoff frequency, the first cutoff frequency being in a range between 0.05 and 0.16 Hz and the second cutoff frequency being in a range between 0.70 Hz and 0.83 Hz. These ranges for the cut-off frequencies of the bandpass filter (0.05-0.16 Hz for the first cutoff frequency, and 0.70-0.83 Hz for the second cutoff frequency), are selected such that the bandpass filter allows signals that correspond to human breathing to pass through the filter, and attenuates other signals. For example, cutoff frequencies of 0.13 Hz and 0.75 Hz correspond to respiration rates of 8 and 45 breaths per minute, respectively.

In illustrative implementations, a computer performs an algorithm for calculating heart rate. The algorithm includes applying one or more filters to an input signal derived from the sensor data and producing a filtered signal, such that the overall effect of the one of more filters is a bandpass filter. The bandpass filter has a first cutoff frequency and a second cutoff frequency, the first cutoff frequency being in a range between 0.70 and 0.80 Hz and the second cutoff frequency being in a range between 2.30 Hz and 3.30 Hz. These ranges for the cut-off frequencies of the bandpass filter (0.70-0.80 Hz for the first cutoff frequency, and 2.30-3.30 Hz for the second cutoff frequency), are selected such that the bandpass filter allows signals that correspond to human heart beats to pass through the filter, and attenuates other signals. For example, cutoff frequencies of 0.75 Hz and 2.5 Hz correspond to heart rates of 45 and 150 beats per minute, respectively. The input signal may itself be a filtered signal.

### Computers

In illustrative implementations, one or more computers (e.g. computer 121) are programmed and specially adapted: (1) to control the operation of, or interface with, hardware components of a sensor module, including a gyroscope, accelerometer, or camera; (2) to control the operation of, or interface with, hardware components of a wireless transceiver unit; (3) to apply any filter to a signal, including any lowpass, highpass, bandpass, Butterworth, Chebyshev, thresholding or averaging filter; (4) to perform an FFT (fast Fourier transform) algorithm or to otherwise calculate a Fourier transform, including a discrete Fourier transform, of any signal; (5) to analyze a frequency spectrum of a signal, including to detect an amplitude peak in a frequency spectrum of the signal, including a peak that is indicative of periodicity of the signal in the time domain; (6) to perform an algorithm that takes sensor readings (including data gathered by a gyroscope, accelerometer, or camera) as input and that calculates respiration rate, heart rate or heart rate variability; (7) to perform an algorithm for signal processing or signal pre-processing; (8) to perform any other calculation, computation, program, algorithm, computer function or computer task described or implied above; (9) to receive signals indicative of human input; (10) to output signals for controlling transducers for outputting information in human perceivable format; and (11) to process data, to perform computations, to execute any algorithm or software, and to control the read or write of data to and from memory devices. The one or more computers may be in any position or positions within or outside of the support structure (e.g., headband) that houses the sensor module or to which the sensor module is attached. For example, in some cases (a) both the sensor module and a computer are housed in, or attached to, the same support structure; or (b) at least one computer is remote from that support structure. The one or more computers are connected to each other or to other devices either: (a) wirelessly, (b) by wired connection, or (c) by a combination of wired and wireless links.

In exemplary implementations, one or more computers are programmed to perform any and all calculations, computations, programs, algorithms, computer functions and computer tasks described or implied above. For example, in some cases: (a) a machine-accessible medium has instructions encoded thereon that specify steps in a software program; and (b) the computer accesses the instructions encoded on the machine-accessible medium, in order to determine steps to execute in the program. In exemplary implementations, the machine-accessible medium comprises a tangible non-transitory medium. In some cases, the machine-accessible medium comprises (a) a memory unit or (b) an auxiliary memory storage device. For example, in some cases, a control unit in a computer fetches the instructions from memory.

In illustrative implementations, one or more computers execute programs according to instructions encoded in one or more tangible, non-transitory, computer-readable media. For example, in some cases, these instructions comprise instructions for a computer to perform any calculation, computation, program, algorithm, computer function or computer task described or implied above. For example, in some cases, instructions encoded in a tangible, non-transitory, computer-accessible medium comprise instructions for a computer to: (1) to control the operation of, or interface with, hardware components of a sensor module, including a gyroscope, accelerometer, or camera; (2) to control the operation of, or interface with, hardware components of a wireless transceiver unit; (3) to apply any filter to a signal, including any lowpass, highpass, bandpass, Butterworth, Chebyshev, thresholding or averaging filter; (4) to perform an FFT (fast Fourier transform) algorithm or to otherwise calculate a Fourier transform, including a discrete Fourier transform, of any signal; (5) to analyze a frequency spectrum of a signal, including to detect an amplitude peak in a frequency spectrum of the signal, including a peak that is indicative of periodicity of the signal in the time domain; (6) to perform an algorithm that takes sensor readings (including data gathered by a gyroscope, accelerometer, or camera) as input and that calculates respiration rate, heart rate or heart rate variability; (7) to perform an algorithm for signal processing or signal pre-processing; (8) to perform any other calculation, computation, program, algorithm, computer function or computer task described or implied above; (9) to receive signals indicative of human input; (10) to output signals for controlling transducers for outputting information in human perceivable format; and (11) to process data, to perform computations, to execute any algorithm or software, and to control the read or write of data to and from memory devices.

### Network Communication

In illustrative implementations of this invention, an electronic device (e.g., gyroscope, accelerometer, camera, other sensor, or computer) is configured for wireless or wired communication with other electronic devices in a network.

For example, in some cases, one or more of the following hardware components are used for network communication: a computer bus, a computer port, network connection, network interface device, host adapter, wireless module, wireless card, signal processor, modem, router, computer port, cables or wiring.

In some cases, one or more computers (e.g., onboard the same support structure as the sensor module) are programmed for communication over a network. For example, in some cases, one or more computers are programmed for network communication: (a) in accordance with the Internet Protocol Suite, or (b) in accordance with any other industry standard for communication, including any USB standard, ethernet standard (e.g., IEEE 802.3), token ring standard (e.g., IEEE 802.5), wireless standard (including IEEE 802.11 (wi-fi), IEEE 802.15 (bluetooth/zigbee), IEEE 802.16, IEEE 802.20 and including any mobile phone standard, including GSM (global system for mobile communications), UMTS (universal mobile telecommunication system), CDMA (code division multiple access, including IS-95, IS-2000, and WCDMA), or LTS (long term evolution)), or other IEEE communication standard.

### I/O Devices

In illustrative implementations, the system (including sensor module for measuring heart rate and respiration rate and a computer) includes, or interfaces with, I/O devices. In some cases, electronic devices in the system and all or some of the I/O devices are located onboard a single support structure (such as a headband). Alternatively, one or more the I/O devices are remote from other electronic devices in the system and are connected to the system via a wired or wireless communication link.

For example, in some cases, the I/O devices comprise one or more of the following: touch screens, cameras, microphones, accelerometers, gyroscopes, magnetometers, inertial measurement units, pressure sensors, touch sensors, capacitive sensors, buttons, dials or sliders.

In illustrative implementations, a human inputs data or instructions via one or more I/O devices. The system outputs data or instructions (including data regarding heart rate, respiration rate or heart rate variability) via one or more I/O devices.

### DEFINITIONS

The terms “a” and “an”, when modifying a noun, do not imply that only one of the noun exists.

“Actual movement” means movement relative to a spatial coordinate system that is not fixed relative to a camera or to points in a scene being imaged by the camera.

To “apply a filter” to a signal means to modify the signal with a filter. For example, in some cases, a filter is applied computationally, or by analog circuitry, or by a combination of computations and analog circuitry.

“Bandpass filter” means any combination of one or more filters that, taken together, have the effect of attenuating a signal less in a specified frequency range than at all frequencies above or below the specified frequency range. Here is a non-limiting example of applying a “bandpass filter”: applying a highpass filter and then a lowpass filter (or vice versa) to a signal, where the cutoff frequency of the lowpass filter is greater than the cutoff frequency of the highpass filter.

To compute “based on” specified data means to perform a computation that takes the specified data as an input.

Here are some non-limiting examples of a “camera”: (a) a video camera; (b) a digital camera; (c) an optical instrument that records images; (d) a depth-sensing camera; (e) a light field camera; or (f) an imaging system. The term “camera” includes any computers that process data captured by the camera.

The term “comprise” (and grammatical variations thereof) shall be construed as if followed by “without limitation”. If A comprises B, then A includes B and may include other things.

The term “computer” includes any computational device that performs logical and arithmetic operations. For example, in some cases, a “computer” comprises an electronic computational device, such as an integrated circuit, a microprocessor, a mobile computing device, a laptop computer, a tablet computer, a personal computer, or a mainframe computer. In some cases, a “computer” comprises: (a) a central processing unit, (b) an ALU (arithmetic/logic unit), (c) a memory unit, and (d) a control unit that controls actions of other components of the computer so that encoded steps of a program are executed in a sequence. In some cases, a “computer” also includes peripheral units including an auxiliary memory storage device (e.g., a disk drive or flash memory), or includes signal processing circuitry. However, a human is not a “computer”, as that term is used herein.

“Defined Term” means a term or phrase that is set forth in quotation marks in this Definitions section.

For an event to occur “during” a time period, it is not necessary that the event occur throughout the entire time period. For example, an event that occurs during only a portion of a given time period occurs “during” the given time period.

The term “e.g.” means for example.

The fact that an “example” or multiple examples of something are given does not imply that they are the only instances of that thing. An example (or a group of examples) is merely a non-exhaustive and non-limiting illustration.

Unless the context clearly indicates otherwise: (1) a phrase that includes “a first” thing and “a second” thing does not imply an order of the two things (or that there are only two of the things); and (2) such a phrase is simply a way of identifying the two things, respectively, so that they each may be referred to later with specificity (e.g., by referring to “the first” thing and “the second” thing later). For example, unless the context clearly indicates otherwise, if an equation has a first term and a second term, then the equation may (or may not) have more than two terms, and the first term may occur before or after the second term in the equation. A phrase that includes a “third” thing, a “fourth” thing and so on shall be construed in like manner.

The term “for instance” means for example.

Non-limiting examples of a “gyroscope” include: (a) a gyroscope with a mass that spins repeatedly about an axis; (b) an analog gyroscope; (c) a digital gyroscope, a digital read-out gyroscope; (d) a digital MEMS (microelectromechanical system) gyroscope; (e) a digital gyroscope that includes one or more piezeoelectric, piezoresistive or capacitive sensors; (f) a single-axis gyroscope, including a single-axis gyroscope of a type described in clauses (a) to (e) of this sentence; and (g) a tri-axial gyroscope, including a tri-axial gyroscope of a type described in clauses (a) to (e) of this sentence. In many cases, a “gyroscope” does not have a mass that spins about an axis in such a way as to complete multiple, 360 degree, spins about the axis.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

“I/O device” means an input/output device. For example, an I/O device includes any device for (a) receiving input from a human, (b) providing output to a human, or (c) both. For example, an I/O device includes a user interface, graphical user interface, keyboard, mouse, touch screen, microphone, handheld controller, display screen, speaker, or projector for projecting a visual display. Also, for example, an I/O device includes any device (e.g., button, dial, knob, slider or haptic transducer) for receiving input from, or providing output to, a human.

The term “or” is inclusive, not exclusive. For example A or B is true if A is true, or B is true, or both A or B are true. Also, for example, a calculation of A or B means a calculation of A, or a calculation of B, or a calculation of A and B.

A parenthesis is simply to make text easier to read, by indicating a grouping of words. A parenthesis does not mean that the parenthetical material is optional or may be ignored.

“Program” means a sequence of steps executed by a computer.

“Rotational motion sensor” means a sensor for measuring rotational motion, which sensor is neither a camera nor a light sensor. Non-limiting examples of a rotational motion sensor include a gyroscope and a magnetometer.

“Some” means one or more.

“Substantially” means at least ten percent. For example: (a) 112 is substantially larger than 100; and (b) 108 is not substantially larger than 100.

The term “such as” means for example.

Spatially relative terms such as “under”, “below”, “above”, “over”, “upper”, “lower”, and the like, are used for ease of description to explain the positioning of one element relative to another. The terms are intended to encompass different orientations of an object in addition to different orientations than those depicted in the figures.

“Visual context” means an object, event or state that exists or occurs in a scene and that is observable in one or more images of the scene captured by a camera. However, “visual context” does not include any motion caused by respiration, by heart beat or by blood moved by a heartbeat.

Except to the extent that the context clearly requires otherwise, if steps in a method are described herein, then the method includes variations in which: (1) steps in the method occur in any order or sequence, including any order or sequence different than that described; (2) any step or steps in the method occurs more than once; (3) different steps, out of the steps in the method, occur a different number of times during the method, (4) any combination of steps in the method is done in parallel or serially; (5) any step or steps in the method is performed iteratively; (6) a given step in the method is applied to the same thing each time that the given step occurs or is applied to different things each time that the given step occurs; or (7) the method includes other steps, in addition to the steps described.

This Definitions section shall, in all cases, control over and override any other definition of the Defined Terms. For example, the definitions of Defined Terms set forth in this Definitions section override common usage or any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. To the extent that any term or phrase is defined or clarified herein, such definition or clarification applies to any grammatical variation of such term or phrase, taking into account the difference in grammatical form. For example, the grammatical variations include noun, verb, participle, adjective, and possessive forms, and different declensions, and different tenses. In each case described in this paragraph, Applicant is acting as Applicant's own lexicographer.

### VARIATIONS

This invention may be implemented in many different ways. Here are some non-limiting examples:

In one aspect, this invention is a method comprising, in combination: (a) a gyroscope gathering data indicative of rotational motion of a head of a human; and (b) one or more computers taking the data as input and calculating a cardiac pulse rate of the human; wherein the gyroscope is housed in, or attached to, headwear that is worn on the head of the user. In some cases, the motion is caused by forces that are transmitted, at least in part, from the chest of the human, through the neck of the human, and to the head. In some cases, the motion is caused, at least in part, by respiration of the human, by heart beats of the human, or by blood flow caused by the heart beats. In some cases, the gyroscope measures rotational motion of the forehead of the human. In some cases: (a) the gyroscope, an accelerometer and a camera gather sensor data indicative of motion of the head of a human; (b) the one or more computers take the sensor data as input and calculate a cardiac pulse rate of the human and a respiration rate of the human; and (c) the gyroscope, accelerometer and camera are each housed in, or attached to, the headwear. In some cases, the one or more computers also calculate heart rate variability. In some cases: (a) the method involves applying one or more filters to a signal derived from the sensor data and producing a filtered signal, such that the overall effect of the one of more filters is a bandpass filter, which bandpass filter has a first cutoff frequency and a second cutoff frequency, the first cutoff frequency being in a range between 0.05 and 0.16 Hz and the second cutoff frequency being in a range between 0.70 Hz and 0.83 Hz; and (b) the one or more computers use data indicative of the filtered signal to calculate respiration rate. In some cases: (a) the method involves applying one or more filters to an input signal derived from the sensor data and producing an output signal, such that the overall effect of the one of more filters is a first bandpass filter, which bandpass filter has a first cutoff frequency and a second cutoff frequency, the first cutoff frequency being in a range between 0.70 and 0.80 Hz and the second cutoff frequency being in a range between 2.30 Hz and 3.30 Hz; and (b) the one or more computers use data indicative of the output signal to calculate heart rate. In some cases, the field of view of the camera overlaps with the field of view of the human user. In some cases: (a) the camera is a video camera; and (b) the one or more computers perform an algorithm that takes, as input, frames captured by the video camera, and that determines motion of a video camera relative to points in a scene that is imaged in the frames. In some cases, the one or more computers: (a) determine a visual context, based on data in video frames captured by the camera during a time period; and (b) associate the visual context with a cardiac pulse rate, respiration rate or heart rate variability measured during the time period. Each of the cases described above in this paragraph is an example of the method described in the first sentence of this paragraph, and is also an example of an embodiment of this invention that may be combined with other embodiments of this invention.

In another aspect, this invention is a system comprising, in combination: (a) a gyroscope for gathering data indicative of rotational motion of a head of a human; and (b) one or more computers for taking the data as input and performing a program to calculate a cardiac pulse rate of the human; wherein the gyroscope is housed in, or attached to, headwear configured for being worn on the head of the user. In some cases, the motion is caused by forces that are transmitted, at least in part, from the chest of the human, through the neck of the human, and to the head. In some cases, the system further comprises a non-transitory, machine-readable medium that has instructions for the program stored on the medium. In some cases in which this machine-readable medium is employed, the rotational motion is rotational motion of the forehead of the human. Each of the cases described above in this paragraph is an example of the system described in the first sentence of this paragraph, and is also an example of an embodiment of this invention that may be combined with other embodiments of this invention.

In another aspect, this invention is a system comprising, in combination: (a) a rotational motion sensor, an accelerometer and a camera for gathering data indicative of motion of a head of a human; and (b) one or more computers for taking the data as input and for performing a program to calculate a cardiac pulse rate of the human and a respiration rate of the human; wherein the rotational motion sensor, accelerometer and camera are each housed in, or attached to, headwear configured for being worn on the head of the user. In some cases, the system further comprises a non-transitory, machine-readable medium that has instructions for the program stored on the medium. In some cases, the motion is caused by forces that are transmitted, at least in part, from the chest of the human, through the neck of the human, and to the head. In some cases, the motion is caused, at least in part, by respiration of the human, by heart beats of the human, or by blood flow caused by the heart beats. In some cases, the rotational motion sensor, accelerometer, camera and at least one of the computers are housed in or attached to elastic headwear. Each of the cases described above in this paragraph is an example of the system described in the first sentence of this paragraph, and is also an example of an embodiment of this invention that may be combined with other embodiments of this invention.

The above description (including without limitation any attached drawings and figures) describes illustrative implementations of the invention. However, the invention may be implemented in other ways. The methods and apparatus which are described above are merely illustrative applications of the principles of the invention. Other arrangements, methods, modifications, and substitutions by one of ordinary skill in the art are therefore also within the scope of the present invention. Numerous modifications may be made by those skilled in the art without departing from the scope of the invention. Also, this invention includes without limitation each combination and permutation of one or more of the abovementioned implementations, embodiments and features.

What is claimed is:

1. A method comprising, in combination:
(a) a gyroscope gathering data indicative of rotational motion of a head of a human; and
(b) one or more computers taking the data as input and calculating a cardiac pulse rate of the human;
wherein the gyroscope is housed in, or attached to, headwear that is worn on the head of the user.

2. The method of claim 1, wherein the motion is caused by forces that are transmitted, at least in part, from the chest of the human, through the neck of the human, and to the head.

3. The method of claim 1, wherein the motion is caused, at least in part, by respiration of the human, by heart beats of the human, or by blood flow caused by the heart beats.

4. The method of claim 1, wherein the gyroscope measures rotational motion of the forehead of the human.

5. The method of claim 1, wherein
(a) the gyroscope, an accelerometer and a camera gather sensor data indicative of motion of the head of a human; and
(b) the one or more computers take the sensor data as input and calculate a cardiac pulse rate of the human and a respiration rate of the human;
wherein the gyroscope, accelerometer and camera are each housed in, or attached to, the headwear.

6. The method of claim 5, wherein the one or more computers also calculate heart rate variability.

7. The method of claim 5, wherein:
(a) the method involves applying one or more filters to a signal derived from the sensor data and producing a filtered signal, such that the overall effect of the one of more filters is a bandpass filter, which bandpass filter has a first cutoff frequency and a second cutoff frequency, the first cutoff frequency being in a range between 0.05 and 0.16 Hz and the second cutoff frequency being in a range between 0.70 Hz and 0.83 Hz; and
(b) the one or more computers use data indicative of the filtered signal to calculate respiration rate.

8. The method of claim 1, wherein:
(a) the method involves applying one or more filters to an input signal derived from the sensor data and producing an output signal, such that the overall effect of the one of more filters is a first bandpass filter, which bandpass filter has a first cutoff frequency and a second cutoff frequency, the first cutoff frequency being in a range between 0.70 and 0.80 Hz and the second cutoff frequency being in a range between 2.30 Hz and 3.30 Hz; and
(b) the one or more computers use data indicative of the output signal to calculate heart rate.

9. The method of claim 5, wherein the field of view of the camera overlaps with the field of view of the human user.

10. The method of claim 5, wherein:
(a) the camera is a video camera; and
(b) the one or more computers perform an algorithm that takes, as input, frames captured by the video camera, and that determines motion of a video camera relative to points in a scene that is imaged in the frames.

11. The method of claim 10, wherein the one or more computers:
(a) determine a visual context, based on data in video frames captured by the camera during a time period; and
(b) associate the visual context with a cardiac pulse rate, respiration rate or heart rate variability measured during the time period.

12. A system comprising, in combination:
(a) a gyroscope for gathering data indicative of rotational motion of a head of a human; and
(b) one or more computers for taking the data as input and performing a program to calculate a cardiac pulse rate of the human;
wherein the gyroscope is housed in, or attached to, headwear configured for being worn on the head of the user.

13. The system of claim 12, wherein the motion is caused by forces that are transmitted, at least in part, from the chest of the human, through the neck of the human, and to the head.

14. The system of claim 12, further comprising a non-transitory, machine-readable medium that has instructions for the program stored on the medium.

15. The system of claim 12, wherein the rotational motion is rotational motion of the forehead of the human.

16. A system comprising, in combination:
(a) a rotational motion sensor, an accelerometer and a camera for gathering data indicative of motion of a head of a human; and
(b) one or more computers for taking the data as input and for performing a program to calculate a cardiac pulse rate of the human and a respiration rate of the human;
wherein the rotational motion sensor, accelerometer and camera are each housed in, or attached to, headwear configured for being worn on the head of the user.

17. The system of claim 1, further comprising a non-transitory, machine-readable medium that has instructions for the program stored on the medium.

18. The system of claim 17, wherein the motion is caused by forces that are transmitted, at least in part, from the chest of the human, through the neck of the human, and to the head.

19. The system of claim 17, wherein the motion is caused, at least in part, by respiration of the human, by heart beats of the human, or by blood flow caused by the heart beats.

20. The system of claim 17, wherein the rotational motion sensor, accelerometer, camera and at least one of the computers are housed in or attached to elastic headwear.
